(19) United States
(12) Patent Application Publication
(10) Pub. No.: US 2017/0342456 A1
(43) Pub. Date: Nov. 30, 2017

(54) METHOD OF DETECTING INVASIVE FUNGI ACCORDING TO MORPHOLOGY THEREOF BASED ON CONTRAST STAINING, AND KIT FOR SAME

(71) Applicant: Shun Cui, Wuhan (CN)

(72) Inventor: Shun Cui, Wuhan (CN)

(21) Appl. No.: 15/678,144

(22) Filed: Aug. 16, 2017

(30) Foreign Application Priority Data
Jun. 22, 2017 (CN) 201710481814.5

(51) Int. Cl. C12Q 1/04; G01N 1/30; G01N 15/14; A61B 5/15

(52) U.S. Cl. CPC C12Q 1/04; A61B 5/150755; G01N 1/30; G01N 15/1456; A61B 5/15003; G01N 2015/1497; G01N 2333/37; G01N 2800/26; G01N 2001/302

(57) ABSTRACT

A method of detecting invasive fungi according to morphology thereof based on contrast staining, including: sterilizing and storing the necessary equipment aseptically; drawing 1 ml of venous blood from a tested subject's elbow vein; dripping one drop of the venous blood, prior to coagulation, into an ampoule containing 0.8 ml of a detection reagent under an aseptic environment; gently shaking the ampoule until the drop of venous blood is evenly distributed; leaving the ampoule to stand for 20 minutes to form a stained solution; sterilizing or disinfecting a microscope slide and a cover slip; dripping one drop of the stained solution on the microscope slide prepared under aseptic condition; observing the sample sequentially with 4×, 10× and 40× objective lenses and a 100× oil-immersion lens; magnifying with a 5 million pixel eyepiece; displaying an image of the sample on computer screen using a high-resolution imaging software for observation and record.

### CROSS-REFERENCE TO RELATED APPLICATION

The present application claims the benefit of Chinese Patent Application No. 201710481814.5 filed on Jun. 22, 2017, the entire contents of which is hereby incorporated by reference.

### FIELD OF TECHNOLOGY

The present application relates to a method of detecting invasive fungi; and more particularly, a rapid method of detecting invasive fungi according to morphology thereof based on contrast staining. The present application also relates to a kit for rapidly detecting invasive fungi according to morphology thereof based on contrast staining.

### BACKGROUND

With the constant deterioration of environmental pollution in recent years, human population infected by fungi is also expanding. Fungal infection has developed from secondary infection in the human population in the past to primary fungal infection in the “normal” population nowadays (evoked by factors such as environment, occupation and fatigue). Furthermore, patients with underlying diseases (for example those with diabetes) and the elderly are both populations susceptible to fungal infection. The widespread application of treatments by the modern interventional technology and surgery has also provided conditions for fungal infection.

There are currently four methods of detecting invasive fungi, namely, morphological detection by direct smear of body fluid; serological detection; pathogen genetic detection; and pathological detection.

The morphological detection by direct smear of body fluid is currently the major method of detecting invasive fungi, which primarily relies on the availability of body fluids or secretions for smear detection and culture identification. However, since the obtained body fluids or secretions are derived from an internal body surface (i.e., a luminal mucosa) which is in communication with the outside world, the obtained samples cannot be isolated from the atmosphere and are easily contaminated. This leads to poor specificity of the detection results and cannot reflect the true situation of the invasive mycosis. Moreover, fungal culture is time-consuming which generally requires more than two weeks, and positive rate of the culture is extremely low. Therefore, rapid detection which facilitates subsequent preemptive clinical treatment is not possible.

Serological detection, including serum galactomannan detection (abbreviated as the GM test) and 1,3-β-D-glucan detection (abbreviated as the G test), has currently been adopted in the clinical detection of invasive fungal infection and the monitoring of treatment response. This provides a rapid and effective basis for the preemptive clinical anti-fungal treatment. However, since its application is influenced by a variety of factors, serological detection is prone to false positive. Moreover, the sensitivity and detection coverage of the types of fungi are limited. Furthermore, serological detection is bound by hospital condition and economy condition of the patient. This makes the detection of invasive fungal infection difficult.

Pathogen genetic detection is also influenced by a variety of factors. Due to its high false positive rate, pathogen genetic detection cannot be the gold standard of diagnosis.

For pathological detection, traumatic means is often required to obtain the sample. As such, pathological detection is rarely adopted clinically.

### SUMMARY

The first objective of the present application is to overcome the deficiency in the prior art, by providing a rapid method of detecting invasive fungi according to morphology thereof based on contrast staining.

The second objective of the present application is to overcome the deficiency in the prior art, by providing a kit for rapidly detecting invasive fungi according to morphology thereof based on contrast staining.

In order to achieve the first objective, the present application provides the following technical solution:

A method of detecting invasive fungi according to morphology thereof based on contrast staining, including:

Step 1: sterilizing equipment necessary for the detection of the invasive fungi; and storing the equipment aseptically;
Step 2: drawing about 1 ml of venous blood from an elbow vein of a tested subject using a 1-2 ml standardized syringe;
Step 3: dripping one drop of the venous blood, prior to coagulation, into a 5 ml ampoule containing 0.8 ml of a detection reagent under an aseptic environment; gently shaking the 5 ml ampoule until the drop of the venous blood is evenly distributed within the 5 ml ampoule; and leaving the 5 ml ampoule to stand for 20 minutes to allow the detection reagent to stain the drop of the venous blood to form a stained solution;
wherein the detection reagent is prepared by:
dissolving 0.8-1.5 g of C.I. Direct Blue 1 powder in 100 ml of aseptic physiological saline to form a C.I. Direct Blue 1 stain solution; filtering and disinfecting the C.I. Direct Blue 1 stain solution by a disposable needle filter; placing the C.I. Direct Blue 1 stain solution in a sterile ampoule; and dispensing and packing the C.I. Direct Blue 1 stain solution into 5 ml ampoules in an amount of 0.8 ml per ampoule;
   Step 4: sterilizing or disinfecting a microscope slide and a cover slip;
wherein the step of sterilizing or disinfecting the microscope slide and the cover slip is performed according to either one of the following:
i. immersing the microscope slide and the cover slip in a 75% alcohol solution for more than 3 hours; heating the microscope slide and the cover slip with an alcohol burner for disinfection at the time of slide preparation; and cooling the microscope slide and the cover slip in an aseptic sealed container with a diameter of greater than 100 mm until the staining in step 3 is completed; or
ii. directly disinfecting the microscope slide and the cover slip on a clean bench with ultraviolet light; and storing the microscope slide and the cover slip aseptically until the staining in step 3 is completed;
   Step 5: drawing the stained solution from the 5 ml ampoule by a 1 ml standardized disposable sterile syringe or sterile dropper; dripping one drop of the stained solution on the microscope slide prepared in step 4 under aseptic condition; and placing the cover slip over the microscope slide; and
   Step 6: upon placing the cover slip over the microscope slide, observing the drop of the stained solution on the microscope slide sequentially with a 4× objective lens, a 10× objective lens, a 40× objective lens, and a 100× oil-immersion lens; magnifying with a 5 million pixel eyepiece; displaying an image of the drop of the stained solution on the microscope slide on a screen of a computer using a high-resolution imaging software; and observing and recording the drop of the stained solution on the microscope slide on the computer.

In order to achieve the second objective, the present application provides the following technical solutions:

A kit detecting invasive fungi according to morphology thereof based on contrast staining, including a detection reagent formed by mixing a C.I. Direct Blue 1 powder with an aseptic physiological saline in a weight ratio of (0.8-1.5):100.

In a preferred embodiment, the detection reagent is formed by mixing a C.I. Direct Blue 1 powder with an aseptic physiological saline in a weight ratio of 1:100.

The existing methods for the detection of invasive fungi are all defective. Meanwhile, since fungi are eukaryotes, morphological detection is the major method of detecting fungi. An effective method with high specificity for the detection of invasive fungi according to morphology thereof is currently lacking due to the following reasons:
1. Traditional clinical thinking lacks the understanding that blood infection must occur during the process where fungi from the outside world invade the human body. Rather, the traditional clinical thinking is that identifying fungi in the blood is like looking for a needle in a haystack. As such, the study of the morphological detection of invasive fungi in the blood is overlooked.
2. The positive rate of fungal blood culture is extremely low. This is also the reason why the fungemia process of invasive fungal infection has been overlooked clinically for a long time, which has deepened the inherent thinking that the possibility of identifying fungi in the blood is extremely low.
3. The existing morphological detection of cells in the blood and body fluid detects static cell morphology after staining and smearing the peripheral blood or body fluids. The detection range thereof is limited to finite types of normal or morbid blood cells (such as leukemia cells) and a small number of microorganisms (such as Plasmodium and Histoplasma). Furthermore, due to the extremely low positive rate of invasive fungal culture, it is not possible to correspond invasive fungi to the cells and related structures observed by the existing smearing methods based on the morphology of static cells. This leads to difficulties in morphological identification under the microscope.
4. The existing smearing methods in cytological detection are operated under non-sterile conditions. Contamination of the smear is inevitable, which also causes the creditability of the smear results in fungal detection to be not high.
5. More importantly, in the course of studying invasive fungal infection over a long period of time, the inventor of the present application has discovered that the incidence of fungal infections is much higher than that recognized by the current clinical thinking. By observing the living cells in the blood, fungal entity and related structures (such as hyphae, sporozoites and fungal balls) can be very easily and clear discovered and identified. However, since the compositions of fungal entity are similar to those of normal cells in the human body (such as erythrocytes and platelets), the staining methods used in the existing cytological smear detection cannot distinguish fungal entity from normal cells in human (such as erythrocytes and platelets) in the static cell detection. This is also one of the important reasons as to why fungal infection is overlooked in existing clinical thinking, leading to the difficulties in the detection of invasive fungal infection.

Comparing with the prior art, the beneficial effects of the present application are as follows:
1. Comparing with various existing methods for fungal detection, the detection time required by the method and kit according to the present application is short. The accuracy is high. The contours of the cells and their related structures are clear. The difference in color depth is clear. The hyphae, spores, spikes on the surface of erythrocytes, etc. may be clearly displayed. The present application provides a simple, quick, and effective method and kit for the detection of fungi based on morphology thereof, and may be promoted to the primary hospitals.
2. The method and the kit according to the present application specifically detect invasive fungal infection based on the specificity in contrast staining of the fungal hyphae in the infected blood and body fluids, and the dynamic detection of living fungal entity and the related structures (such as hyphae and spores) in the blood and body fluids.
3. Since the method and the kit according to the present application may quickly and accurately assist the clinician to determine whether or not the tested subject is infected with invasive fungi, and meanwhile the existing method of detecting invasive fungi still lacks a rapid blood morphology detection, and further in view of the relatively high mortality rate of invasive fungal infection (the mortality rate being 11% if the antifungal therapy is administered within 12 hours, and the mortality rate being 33% if the anti-fungal therapy is initiated after 12 hours), the present application possesses good social benefits and market prospect.
4. With the support of computer aided device (high-resolution electrophotographic apparatus), the method and the kit according to the present application enables the staff to clearly observe various tangible components in the blood under the microscope, which are different to observe by the naked eye. This may provide a reliable basis for determining an accurate and rapid clinical treatment. Also, the various tangible components in the blood not only include human blood cells and their functional states under normal and pathological conditions, as well as atypical eukaryotic microbial cells, but also the components having various morphology and properties not visible to the naked eye which lead to blood circulatory disorder, such as thrombus, fungal embolus, floating hyphae (and fungal balls), and various anchoring structures (anchoring hyphae, budding spores, etc.). Since the method according to the present application is operated under aseptic condition, it may objectively reflect the true situation of invasive fungal infection in the body.
5. Since the method and the kit according to the present application may identify microscopic fungal embolus under the microscope which are not visible to the naked eye, it may rapidly detect microscopic fungal embolus or thrombus that may cause microcirculatory disorder, before anchored embolus which is visible to the naked eye is identified. Meanwhile, the method and the kit according to the present application may also be applied to the rapid screening of blood donors and the blood test before transfusion, so as to rapidly identify the presence of fungal spores and hyphae in blood transfusion.

### BRIEF DESCRIPTION OF DRAWINGS

FIG. 1 is a flow diagram according to the present application.

FIG. 2 is an image of the blood of a normal human subject (without fungi) after staining by the C.I. Direct Blue 1 stain.

FIG. 3 is an image of hyphae without staining.

FIG. 4 is an image of hyphae after staining by the C.I. Direct Blue 1 stain.

FIG. 5 is an image of a sample of body fluid culture under the microscope in a morphological detection of fungi. The sample was cultured for two weeks and directly smeared.

FIG. 6 is an image of a sample of body fluid culture which was obtained from the same individual and prepared in the same way as that shown in FIG. 5, except that this sample was subjected to the detection method according to the present application.

FIG. 7 is an image of a blood sample from an individual who had been confirmed to carry fungi in the blood. The sample was subjected to the detection method according to the present application.

FIG. 8 is an image of a body fluid sample in a galactomannan antigen detection (GM test) of fungi.

FIG. 9 is an image of a body fluid sample which was obtained from the same individual and prepared in the same way as that shown in FIG. 8, except that this sample was subjected to the detection method according to the present application.

FIG. 10 is an image obtained by the method of detecting whether fungi are present in the pleural effusion according to the present application.

FIG. 11 is an image obtained by the method of detecting whether fungi are present in the cerebrospinal fluid according to the present application.

FIG. 12 is an image of provoked leukocytes.

In the figures:

1—normal erythrocyte; 2—normal leukocyte; 3—normal platelet; 4—hypha; 5—abnormal erythrocyte; 6—spore and sporozoite; 7—provoked leukocyte; 8—fiber exudates; 9—crystal.

### DETAILED DESCRIPTION

Embodiments of the present application are described below with reference to the accompanying drawings. However, these embodiments are not intended to constitute limitations on the present application, but are merely examples of the present application. Meanwhile, the following description would render the advantages of the present application clearer and easier to understand.

In the following examples, C.I. Direct Blue 1 (CAS No.: 2610-05-1; trade name: Chicago Sky Blue; Product No.: Sigma—C8679) was purchased from Sigma-Aldrich Trading Co., Ltd. (Shanghai, China). C.I. Direct Blue 1 has a chemical formula of:

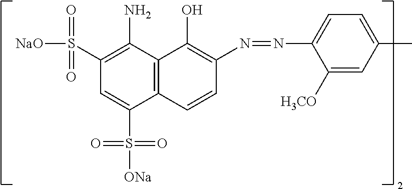

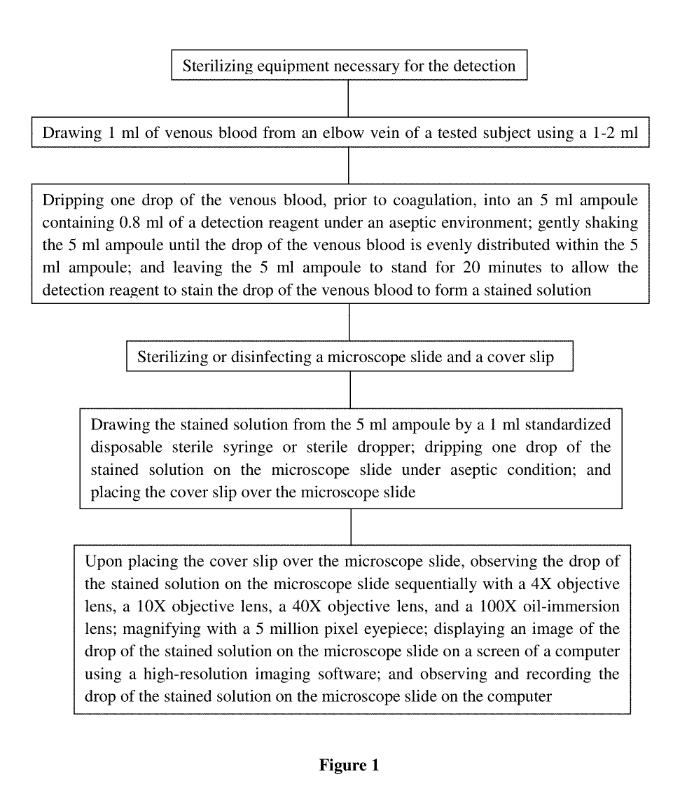

Referring to FIG. 1, the present application provides a method of detecting invasive fungi according to morphology thereof based on contrast staining, including:

Step 1: sterilizing equipment necessary for the detection of the invasive fungi; and storing the equipment aseptically;
Step 2: drawing about 1 ml of venous blood from an elbow vein of a tested subject using a 1-2 ml standardized syringe;
Step 3: dripping one drop of the venous blood, prior to coagulation, into a 5 ml ampoule containing 0.8 ml of a detection reagent (diluted at 15-fold) under an aseptic environment; gently shaking the 5 ml ampoule until the drop of the venous blood is evenly distributed within the 5 ml ampoule; and leaving the 5 ml ampoule to stand for 20 minutes to allow the detection reagent to stain the drop of the venous blood to form a stained solution;
wherein the detection reagent is prepared by:
dissolving 0.8-1.5 g of C.I. Direct Blue 1 powder in 100 ml of aseptic physiological saline to form a C.I. Direct Blue 1 stain solution; filtering and disinfecting the C.I. Direct Blue 1 stain solution by a disposable needle filter; placing the C.I. Direct Blue 1 stain solution in a sterile ampoule; and dispensing and packing the C.I. Direct Blue 1 stain solution into 5 ml ampoules in an amount of 0.8 ml per ampoule;
   Step 4: sterilizing or disinfecting a microscope slide and a cover slip;
wherein the step of sterilizing or disinfecting the microscope slide and the cover slip is performed according to either one of the following:
iii. immersing the microscope slide and the cover slip in a 75% alcohol solution for more than 3 hours; heating the microscope slide and the cover slip with an alcohol burner for disinfection at the time of slide preparation; and cooling the microscope slide and the cover slip in an aseptic sealed container with a diameter of greater than 100 mm until the staining in step 3 is completed; or
iv. directly disinfecting the microscope slide and the cover slip on a clean bench with ultraviolet light; and storing the microscope slide and the cover slip aseptically until the staining in step 3 is completed;
   Step 5: drawing the stained solution from the 5 ml ampoule by a 1 ml standardized disposable sterile syringe or sterile dropper; dripping one drop of the stained solution on the microscope slide prepared in step 4 under aseptic condition; and placing the cover slip over the microscope slide; and
   Step 6: upon placing the cover slip over the microscope slide, observing the drop of the stained solution on the microscope slide sequentially with a 4× objective lens, a 10× objective lens, a 40× objective lens, and a 100× oil-immersion lens; magnifying with a 5 million pixel eyepiece; displaying an image of the drop of the stained solution on the microscope slide on a screen of a computer using a high-resolution imaging software; and observing and recording the drop of the stained solution on the microscope slide on the computer.

Preferably, the detection reagent is formed by mixing a C.I. Direct Blue 1 powder with the aseptic physiological saline in a weight ratio of (0.8-1.5):100. More preferably, the detection reagent is formed by mixing a C.I. Direct Blue 1 powder with the aseptic physiological saline in a weight ratio of 1:100.

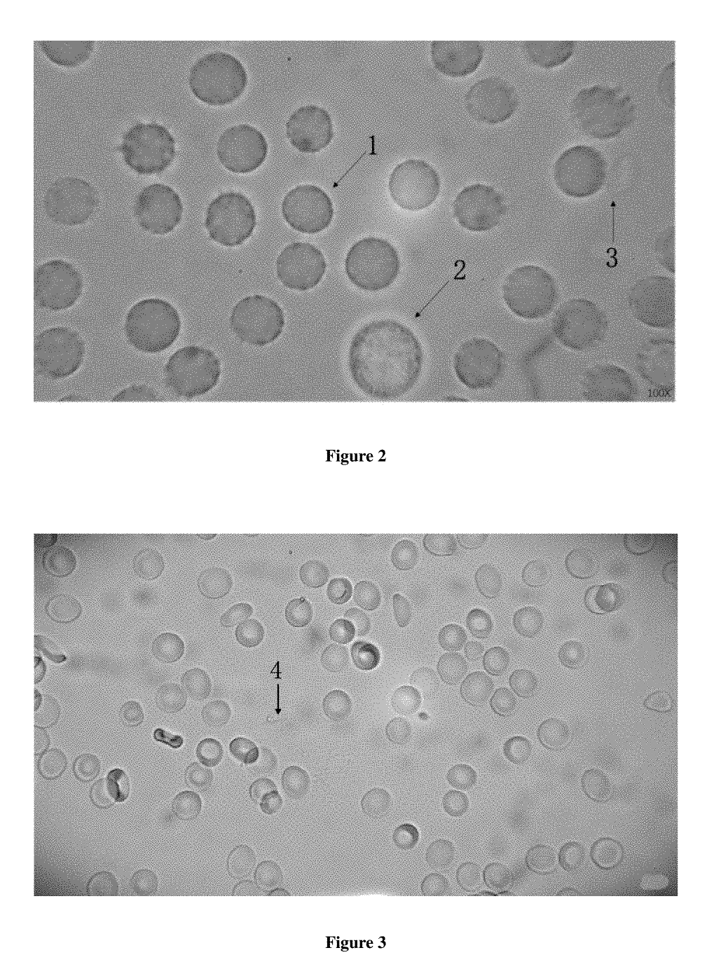

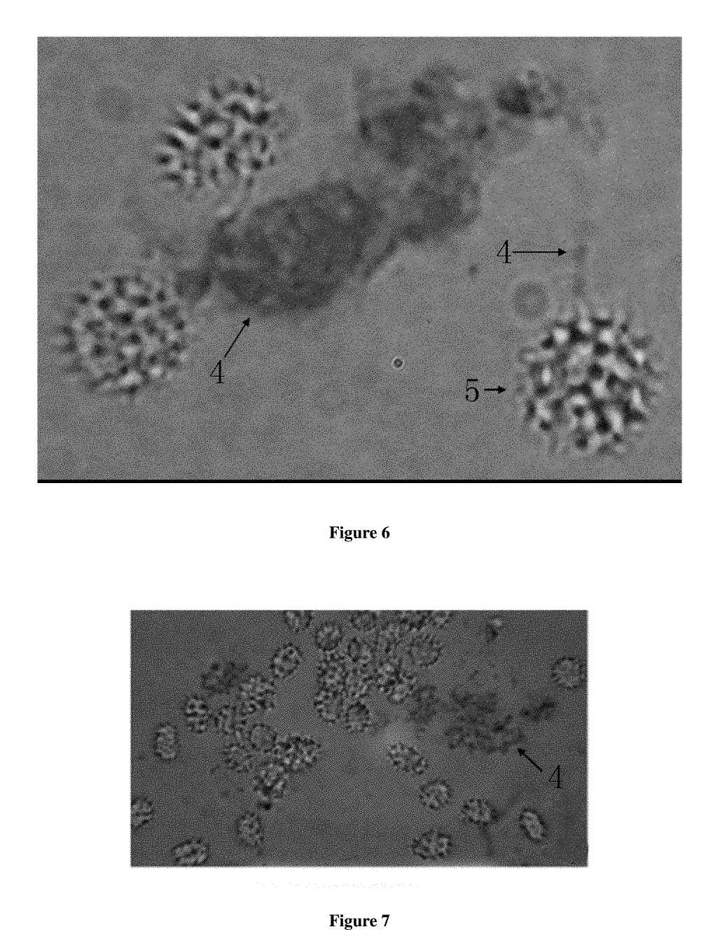

As shown in FIGS. 2, 6 and 7, when being stained by the C.I. Direct Blue 1 stain, significant difference was shown in the morphological features between the normal erythrocytes and the abnormal erythrocytes in the blood. Based on the cell morphology, one skilled in the art may conveniently and rapidly determine whether fungi or spores are present in the blood.

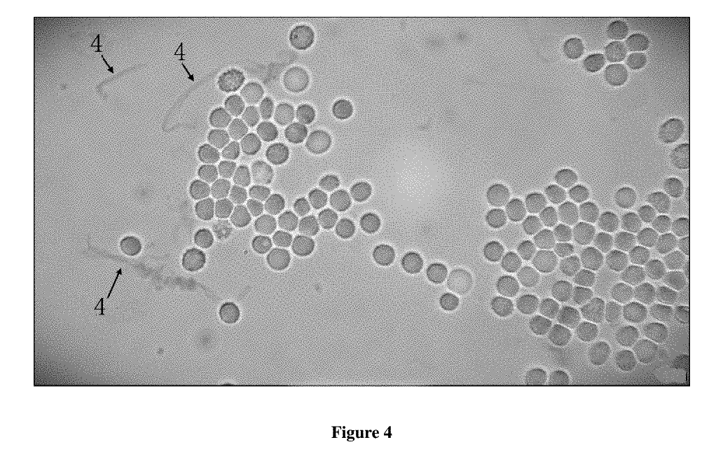

As shown in FIGS. 3 and 4, the difference between the stained hyphae and the non-stained hyphae under the microscope was very significant. Based on the results observed under the microscope, one skilled in the art not only may rapidly determine whether fungi are present in the blood of the tested subject, but also may preliminarily determine the approximate quantity of bacteria in the blood of the tested subject.

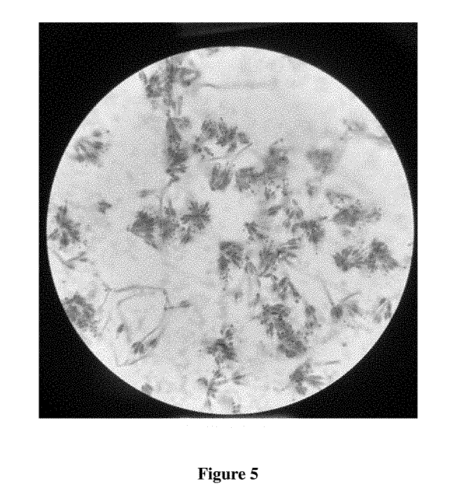

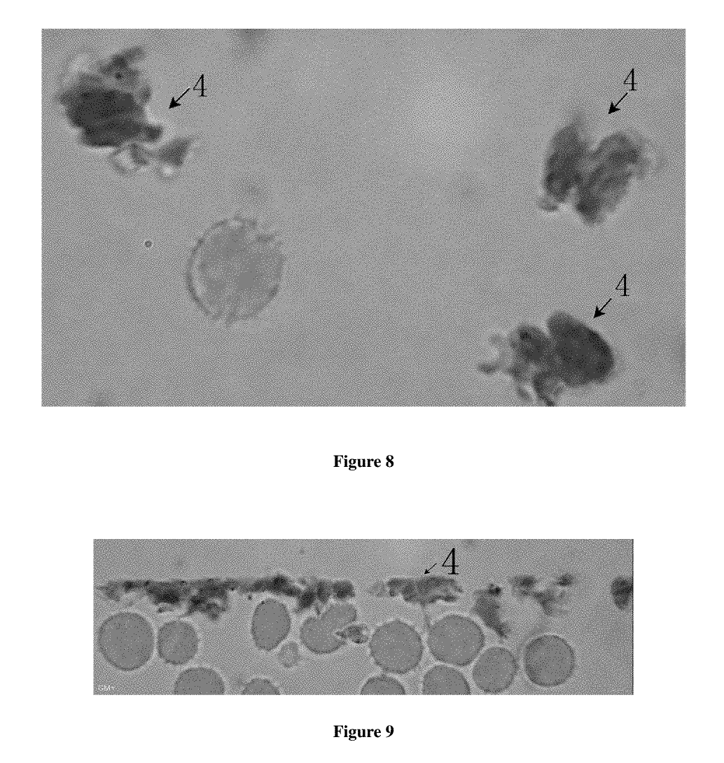

As shown in FIGS. 5, 6, 8 and 9, the method according to the present application was effective in the detection of fungi and might be used for detecting whether fungi are present in the blood. Meanwhile, since the method according to the present application only requires about 20 minutes of staining time to accurately determine whether fungi are present in the tested body fluid, as compared to the various existing detection methods, the method according to the present application may significantly shorten the detection time and cost, increase the detection accuracy, and possess good social benefits and market prospect.

As shown in FIG. 7, the method according to the present application could clearly detect the presence of fungi in the body of the fungal infected subject. Therefore, the method according to the present application is effective in detecting fungi and worthy for popularization.

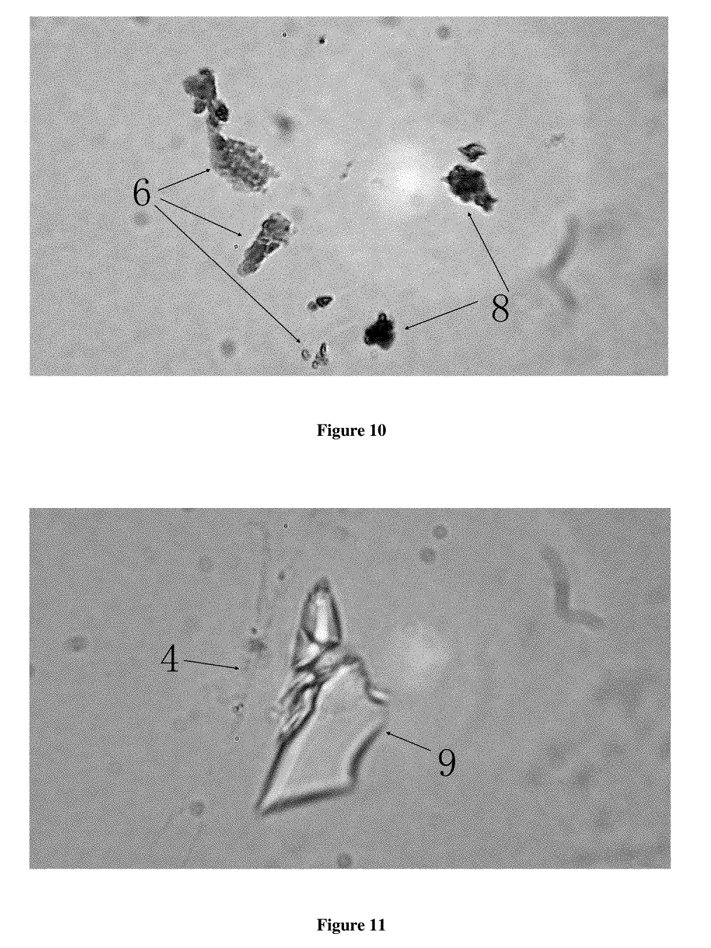

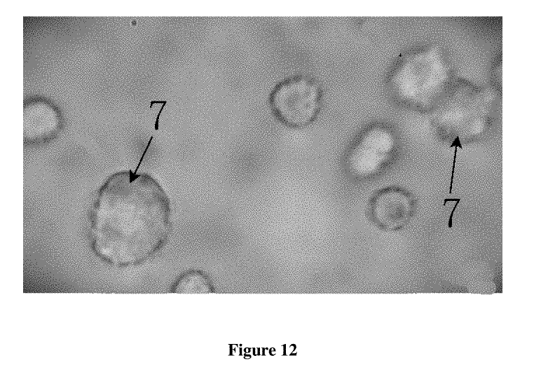

As shown in FIGS. 9, 10, 11 and 12, the method according to the present application might be widely adopted in the detection of fungi. It may be used not only in the detection of whether fungi are present in the blood, but also in the detection of whether fungi are present in other fluids (such as pleural effusion, cerebrospinal fluid, and synovial fluid). Meanwhile, the method according to the present application may also be adopted in other experiments (such as the leukocyte provocation test shown in FIG. 12).

In order to more clearly explain the characteristics of the method and the kit according to the present application, and further illustrate the difference between the samples stained by the C.I. Direct Blue 1 stain and those not stained by the C.I. Direct Blue 1, the inventor of the present application conducted a comparison on the same sample with and without being stained, and the results were as follows:

TABLE 1
Comparison between blood sheet stained by C.I.
Direct Blue 1 contrast stain and blood sheet not
stained by C.I. Direct Blue 1 contrast stain.

| | | Stained by C.I. Direct Blue 1 |
|---|---|---|
| | Not Contrast Stained | Contrast Stain |
| Cell | Uniform color, consistent | Clear cell contours, clear |
| Color | with the color of | difference in color depth |
| | erythrocyte | |
| Hypha | Not visualized | Hyphae and spores could be |
| | | clearly visualized |
| Spore | Sporozoites distributed | Sporozoites distributed at |
| | at the bottom of the | the bottom of the microscope |
| | microscope slide could | slide could be clearly visualized |
| | not be visualized | |
| Infected | Infected leukocytes could | Infected leukocytes could |
| Cell | not be visualized | be visualized |
| Erythro- | Spikes on the surface of | Spikes on the surface of |
| cyte | erythrocytes could not be | erythrocytes could be clearly |
| | visualized | visualized |
| Cell | Statically | Dynamically - various cell |
| Move- | | functional states of the |
| ment | | infected cells could be |
| | | observed dynamically |
| Other | Stainable structures at the | Stainable structures at the |
| | bottom of the microscope | bottom of the microscope |
| | slide could not be | slide could be contrast |
| | visualized | stained and visualized |

Other portions which have not been specifically described in the present application belong to the prior art.

What is claimed is:

1. A method of detecting invasive fungi according to morphology thereof based on contrast staining, comprising:
step 1: sterilizing equipment necessary for the detection of the invasive fungi; and storing the equipment aseptically;
step 2: drawing 1 ml of venous blood from an elbow vein of a tested subject using a 1-2 ml standardized syringe;
step 3: dripping one drop of the venous blood, prior to coagulation, into a 5 ml ampoule containing 0.8 ml of a detection reagent under an aseptic environment; gently shaking the 5 ml ampoule until the drop of the venous blood is evenly distributed within the 5 ml ampoule; and leaving the 5 ml ampoule to stand for 20 minutes to allow the detection reagent to stain the drop of the venous blood to form a stained solution;
wherein the detection reagent is prepared by:
dissolving 0.8-1.5 g of C.I. Direct Blue 1 powder in 100 ml of aseptic physiological saline to form a C.I. Direct Blue 1 stain solution; filtering and disinfecting the C.I. Direct Blue 1 stain solution by a disposable needle filter; placing the C.I. Direct Blue 1 stain solution in a sterile ampoule; and dispensing and packing the C.I. Direct Blue 1 stain solution into 5 ml ampoules in an amount of 0.8 ml per ampoule;
step 4: sterilizing or disinfecting a microscope slide and a cover slip;
wherein the step of sterilizing or disinfecting the microscope slide and the cover slip is performed according to either one of the following:
v. immersing the microscope slide and the cover slip in a 75% alcohol solution for more than 3 hours; heating the microscope slide and the cover slip with an alcohol burner for disinfection at the time of slide preparation; and cooling the microscope slide and the cover slip in an aseptic sealed container with a diameter of greater than 100 mm until the staining in step 3 is completed; or
vi. directly disinfecting the microscope slide and the cover slip on a clean bench with ultraviolet light; and storing the microscope slide and the cover slip aseptically until the staining in step 3 is completed;
step 5: drawing the stained solution from the 5 ml ampoule by a 1 ml standardized disposable sterile syringe or sterile dropper; dripping one drop of the stained solution on the microscope slide prepared in step 4 under aseptic condition; and placing the cover slip over the microscope slide; and
step 6: upon placing the cover slip over the microscope slide, observing the drop of the stained solution on the microscope slide sequentially with a 4× objective lens, a 10× objective lens, a 40× objective lens, and a 100× oil-immersion lens; magnifying with a 5 million pixel eyepiece; displaying an image of the drop of the stained solution on the microscope slide on a screen of a computer using a high-resolution imaging software; and observing and recording the drop of the stained solution on the microscope slide on the computer.

2. A kit detecting invasive fungi according to morphology thereof based on contrast staining, comprising a detection reagent formed by mixing a C.I. Direct Blue 1 powder with an aseptic physiological saline in a weight ratio of (0.8-1.5):100.

3. A kit detecting invasive fungi according to morphology thereof based on contrast staining, comprising a detection reagent formed by mixing a C.I. Direct Blue 1 powder with an aseptic physiological saline in a weight ratio of 1:100.
